(19) United States
(12) Patent Application Publication
(10) Pub. No.: US 2014/0242551 A1
(43) Pub. Date: Aug. 28, 2014

(54) Oral Care System and Method

(71) Applicant: Richard D. Downs, Dubuque, IA (US)

(72) Inventor: Richard D. Downs, Dubuque, IA (US)

(21) Appl. No.: 14/193,258

(22) Filed: Feb. 28, 2014

Related U.S. Application Data
(60) Provisional application No. 61/770,408, filed on Feb. 28, 2013.

(51) Int. Cl. A61C 5/04; A61C 5/02; A61K 6/00; A61C 19/06

(52) U.S. Cl. CPC A61C 5/04; A61C 19/066; A61C 5/023; A61K 6/0035
USPC 433/216; 433/224

(57) ABSTRACT

An oral care system and method for the treatment of certain oral pathological conditions that require the cleaning and cleansing of the underlying scaffolding of the tooth. The oral care system and method generally includes applying chlorine dioxide liquid or gel to a diseased portion of the tooth and activating it so as to clean the aforementioned. A tool is utilized to activate the chlorine dioxide liquid thereby releasing its cleaning action. Another process utilizes repetitive steps of water and chlorine dioxide activated with a tool to cleanse the inner regions of an affected tooth.

### CROSS REFERENCE TO RELATED APPLICATIONS

I hereby claim benefit under Title 35, United States Code, Section 119(e) of U.S. provisional patent application Ser. No. 61/770,408 filed Feb. 28, 2013. The 61/770,408 application is currently pending. The 61/770,408 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to an oral care system and more specifically it relates to an oral care system and method for the treatment of certain oral pathological conditions that require the cleaning and cleansing of the underlying scaffolding of the tooth.

2. Description of the Related Art

Any discussion of the related art throughout the specification should in no way be considered as an admission that such related art is widely known or forms part of common general knowledge in the field.

For the purposes of this disclosure, it should be understood that chlorine dioxide, its chemical formula ClO₂, and the abbreviation “CD” wherever referenced may be used interchangeably, and have the same meaning and chemical structure.

The following comprehensive background on the cause, diagnosis, treatment, and failed treatment of periodontal disease and root canals, illustrates the many dimensions dentists must consider when performing periodontal and endodontic procedures. There are many problems currently associated with these procedures including but not limited to (1) inadequate disinfecting methods and or solutions regardless of the level of skill with which they are applied, (2) the dangers related to disinfecting solutions that are considered the standard of dental care, (3) the commercial losses suffered by dentists, and (4) injuries caused to patients as a result of these problems, inadequacies and inefficiencies.

### Current Technology.

Approximately 20,000,000 root canal operations are performed annually in the United States alone. According to statistics gathered by the United States Surgeon General, most adults show signs of periodontal or gingival diseases. Further, severe periodontal disease that needs some form of treatment affects about 14 percent of adults aged 45 to 54 in the US alone.

Periodontitis, an advanced state of gum disease is preceded by the earlier stages of gum disease and gingivitis. These illnesses are caused by bacteria and other pathogens that work along with mucus and other particles to constantly form a sticky, colorless ‘plaque’ on teeth. This plaque, more accurately referred to as a biofilm in the literature, is daily treated by brushing and flossing that can help reduce its growth. However, plaque that is not removed can harden and form calculus (‘tartar’) that brushing alone cannot clean. Thus, individuals are required to seek the services of a dentist or dental hygienist who can professionally clean and remove tartar buildup. For purposes of understanding, the words “gums” or “gum tissue” may be used as a common general term in this application to refer to periodontal or gingival tissues.

Without a routine cleaning, the biofilm and the more stubborn tartar will get worse as long as they remain on teeth. This has the unfortunate side effect of increasing the likelihood that the bacteria will precipitate an inflammation of the gingiva. This sanguine swelling, otherwise known as ‘gingivitis,’ can even cause the affected tissues to bleed if not treated appropriately. Even worse than gingivitis is the possibility of an individual developing periodontitis.

When gum disease reaches the periodontal stage, the gingiva pulls away from the teeth and form spaces, called periodontal ‘pockets’ that become infected with bacteria. The body's immune system fights the aforementioned as the plaque spreads and grows below the gum line. Bacterial toxins, including endotoxins and volatile organic compounds such as methyl mercaptan, viruses, fungi and the body's natural response to the infection start to break down the bone and connective tissue that hold the teeth in place. Thus, if periodontitis is not treated quickly, the bones, gums, and tissue that support the teeth can be severely damaged leading to the possibility of the eventual removal of affected teeth.

Current treatments of periodontal disease centers on the control of the infection. The number and types of treatments varies, and directly depends on the extent of the gum disease. Typically, once these procedures are utilized the patient is in need of a deep cleaning remedy to his or her illness; amongst the treatments for periodontitis currently in practice are (1) scaling, (2) root planing and (3) laser treatment (4) antimicrobial agents. Scaling means scraping off the calculus from the tooth and root surfaces above, below and inside the free gingival margin. Root planing gets rid of rough spots on the tooth root where the germs gather, and helps remove bacteria that contribute to the disease. Finally, in some cases a laser may be used to remove plaque and tartar, wherein laser procedures can result in less bleeding, swelling, and discomfort compared to traditional deep cleaning methods.

### Root Canal

In some circumstances teeth can become severely damaged; deep tooth decay, repeated dental procedures, and/or large fillings, a crack or chip in the tooth, a trauma to the face, can all cause the tooth nerve and pulp to become irritated, inflamed, and infected. In these cases, a root canal is routinely used to repair and save a tooth that is badly decayed or has become infected. During a root canal procedure, the nerve and pulp are removed and the inside of the tooth is cleaned and sealed. Without treatment, the tissue surrounding the tooth becomes infected and abscesses may form therein.

The traditional root canal process starts with drilling an access hole into the tooth. Once this hole is completed, the pulp along with bacteria, the decayed nerve tissue and related debris is removed from the tooth. A cleaning process is then performed by inserting root canal files through the drilled hole; several of these files of increasing diameter are each subsequently placed into the access hole and worked down the full length of the tooth to scrape and scrub the sides of the root canals.

Wikipedia describes a typical root canal therapy process, but fails to disclose the criticality of the rinse cycle as a foundational step in the process, and further fails to discuss the vital importance of disinfecting prior to filling. Wikipedia (Jan. 31, 2013) illustrated a fundamental three-step process of opening the tooth, removing the root pulp, and obturation of the tooth opening. The textual description gives only passing mention to root canal irrigation by simply listing the irrigants commonly used: 5.25% sodium hypochlorite (NaOCl), 6% sodium hypochlorite with surface modifiers for better flow into nooks and crannies, 2% chlorhexidine gluconate (Perioxidina Plus-2), 0.2% chlorhexidine gluconate plus 0.2% cetrimide (Cetrexidin), 17% ethylenediaminetetraacetic acid (EDTA), Framycetin sulfate (Septomixine), and Biopure MTAD Mixture of citric acid, Doxycycline, phosphoric acid, and Tween-80 (detergent) by Dentsply USA (MTAD).

Similarly, the illustrative process of root canal therapy taught by Encyclopedia Britannica completely ignores the debridement, irrigation and disinfecting process. It is only by following links provided on Encyclopedia Britannica online that reference to rinsing or disinfecting is found, a name on WebMD that provides one reference; “Water or sodium hypochlorite is used periodically to flush away the debris.”

The American Dental Association does provides patient information that describes a six-step root canal process, but gives only passing mention to disinfecting irrigation within one step, stating “Medication may be added to the pulp chamber and root canal(s) to help eliminate bacteria.” Alarmingly, the ADA fails to provide any guidance to dentists stating: “There is no professional/clinical information on this topic.”

Despite the aforementioned shortcomings in the available literature the inventor would like it to be understood that current treatments use water or sodium hypochlorite (NaOCl) and other chemicals to periodically flush away the debris in a root canal procedure. Then once the tooth is thoroughly cleaned it is sealed. Before the tooth is sealed, however, several options are available to dentists. Some of them like to wait a week before sealing the tooth for various reasons. For instance, if there is an infection, a dentist may put a medication inside the tooth to clear it up sealing the tooth at a later date. Others may choose to seal the tooth the same day it is cleaned out.

If on the other hand, the root canal is not completed on the same day, a temporary filling is placed in the exterior tooth hole thereby restricting contaminants like saliva and food from entering therein. The typical final process of the root canal is to fill the interior of the tooth by inserting gutta-percha with a sealer cement or a sealer only into the tooth's root canal. Then the exterior access hole created at the beginning of treatment is closed using a filling placed therein.

The removal of plaque during periodontal procedures, and the removal of nerve tissue and pulp during endodontic procedures is largely a mechanical operation performed by the doctor or hygienist. Several types of hand tools such as files, spatulas, scrapers, curettes, and others are utilized to scrape, pry, or otherwise remove tissue, plaque, calculus and other unwanted material from the procedure site. Additionally, rotational tools such as hand pieces with drills or burrs installed may be used. Despite the effective use of these hand tools dental professional may find it necessary to use lasers, sonic, ultrasonic or photoacoustic instruments that can assist in tissue or material removal. In spite the skill and care of the dentist, there is a practical limit to the ability of mechanical instruments to reach all of the tiny periodontal pockets, or the deep root tips during a root canal.

### Irrigation

Thus, reaching into the otherwise inaccessible periodontal pockets presents a problem for dentists. Current dental technology provides a pressurized irrigation solution; this uses water, a debriding solution or a disinfecting solution to overcome this difficulty. The aforementioned solutions are utilized help in the removal of small particles as well as in the disinfection of the procedure site. As can be understood by those skilled in the art, the importance of infection control throughout endodontic or periodontal procedures is paramount. Thus, irrigation occurs throughout these procedures in order to flush away debris. In particular, root canal irrigation plays an important role in the debridement and disinfection of the root canal system and is an integral part of root canal preparation procedures.

Typical root canal procedures use these irrigants most frequently: NaOCl, hydrogen peroxide, chlorhexidine, EDTA or the combined use of all. These liquids deliver good tissue dissolving and disinfection capabilities and have been widely demonstrated in use over many decades. These solutions, and the irrigation process, are well known to the community of endodontists and periodontists. However, the concentration of the irrigants is still a matter of debate and remains controversial, with most advocating around 5.25% to 6% concentration of sodium hypochlorite, while others advocating a lower concentration. A typical Hydrogen peroxide irrigant has a concentration level of 3%, and EDTA is typically used in a concentration level of 15% to 17%. Finally, as described above the causes, diagnoses, and treatment of these oral care diseases are well known in the dental community, and are generally considered to be the standard of care.

### Failed Procedures

Despite the modern standard of care as well as the high understanding of dental disease and treatment thereof, many patients are injured as a direct result of periodontal or endodontic treatment. Additionally, nearly 15% of endodontic procedures fail in the United States alone. Some of the causes of these failures were anticipated though not necessarily specifically described in the work of a noted medical professional in the early part of the twentieth century.

During the 1920s, Dr. Weston A. Price wrote about dental conditions, specifically, causes of dental decay and physical degeneration as well as the destructive effects of root canals. Although his findings delineated in this very old research were highly important, they are still largely ignored by most professional publications and teaching institutions even to the present day because they were not well controlled studies. Thus, many dentists do not know that bacteria and other infectious organisms are always present in the dentin tubules after root canal surgery. However, new research seems to confirm the earlier research.

Reinforcing Dr. Price's findings, research by Dr. Boyd Haley of the University of Kentucky found that 75% of root canal teeth have residual bacterial infections remaining in the dentinal tubules; thus, these produce toxic waste that enters the blood stream causing adverse systemic affects. Further concurring with Price, Dr. James A. Howenstine, a board certified internal medicine specialist, reported in 2005 that very few dentists are aware of or willing to admit that dentin tubules are always infected after root canal surgery. As a result, these bacteria escape into the blood and proceed to initiate a number of degenerative diseases. Blinding modern dentists to the danger posed to their unsuspecting patients is there inherent belief that the disinfecting substances used to pack the root canal after surgery effectively sterilizes the root canal site which is unfortunately not true.

Howenstine reported that some dentists are wrongly convinced that the removal of pulp and packing the root canal cavity with a disinfecting substance blocks the supply of nutrients to the dentin tubules; thus, without the flow nutrients infection cannot be nourished thereby ensuring eradication of infection. However, there are billions of bacteria in root canal teeth including bacteria which are located nearest to the dentinal surface, but plugged below and within the smear layer, and bacteria located in the lateral accessory root canals and dentinal tubules move into these canals. They then migrate into the hard fibrous membrane that holds the tooth in the socket (periodontal membrane). Once established in the periodontal membrane it is easy for them to spread through this membrane and pass into the surrounding bony network. From the bone structure the bacteria proceed to enter the blood vessels of the mandible. The bacteria then travel via the blood stream to a gland, organ or tissue where they can start a new infection.

Thus a focal infection from a root canal source can spread to a distant site creating a new disease, as found by Drs. Haley, Price and Howenstein. This is simply one example of a systemic medical problem resulting from failure to remove or destroy bacteria in dentine tubules. Thus, if proper disinfection or cleaning is not done during the procedure there is a high risk of the occurrence of a bone infection and/or cyst even after several years. It should also be understood that failed root canal procedures most often result from human error, limitations of inadequate tissue removal, and limitations on the state of the art disinfecting solutions.

In this regard, inadequate disinfection can result in a recurring infection, chronic sickness, cysts, and a number of other maladies which the root canal was intended to correct or prevent. After the dentist uses all mechanical means to clean and disinfect a root canal, they must then rely on chemicals to penetrate further into the canals, killing bacteria, more properly referred to as a chemical debridement.

In the 2004 study “Molecular evaluation of residual endodontic microorganisms after instrumentation, irrigation and medication with either calcium hydroxide or Septomixine”, G Tang and LP Samaranayake, H-K Yip indicate that substances commonly used to clean the root canal space fail to completely sterilize the canal. As an example of this it should be understood that currently, many dentists fill root canals immediately as soon as they believe they have completed the cleaning and shaping. Unfortunately, tissue remnants and infection can be left behind in the root canal system that has not been treated properly; this because it has been demonstrated that allowing adequate time of exposure of bacteria to disinfecting solutions is absolutely necessary to eliminate them completely.

Thus as indicated by the aforementioned professionals, many dentists fail to recognize the presence of the smear layer, microcrystalline and organic particle debris that is found spread on root canal walls after root canal instrumentation. Further, it can be understood from their work that necrotic tissue and bacteria remain behind in the smear layer that can block canals and dentin tubules; this has the effect of allowing for the establishment of new bacterial colonies leading to re-infection. All of this can be made even worse if there are many root canals with curves, as this increases the difficulty of the cleaning and filling process.

These curved spaces complicate the dental procedure sometimes leading to a tool accidentally penetrating the side of the tooth. A hole made by the tool would necessarily also have to be filled properly to prevent further infections through that hole. Regardless, this human error exemplified the criticality of aggressive application of effective disinfecting rinses to prevent the introduction of bacteria into unintended tissue. Because of all of this, it is necessary today to widen canals more than would be desired in order to get more tissue scraped out of the canals. This is problematic when a canal is ribbon shaped making the chemical removal of tissue even more important. Because of the possibility of perforation and the inaccuracy of the mechanical means, it is ideal for canals to receive much less mechanical widening and much more reliance on chemical debridement.

Complications may arise if the doctor fails to detect any cracks in the teeth. Such undetected small cracks may become the gateway for the entry of bacteria and infect the tooth again. It is also more likely to weaken a tooth and make it more susceptible to cracks if the root canal must be widened mechanically to clean the canal. Root canal complications may involve the infection of the tooth after root canal treatment, due to defective dental restoration, broken tools lodged in the canal, lack of microbial toxin removal, poor obturation, or incomplete disinfection.

Therefore, in summary of root canal failures, those skilled in the art recognize that the current standard of care results in (a) inadequate time exposure of bacteria to disinfecting rinses; (b) inadequate penetration of disinfecting solutions into deep or difficult to reach pockets, or primary or transverse canals; (c) ineffectiveness of the disinfecting solutions to kill all the microbes and remove bacterial toxins regardless of the skill with which they are applied; (d) inadequate debridement prior to and after application of the disinfecting rinse; and (e) too much widening and weakening of the root canal hard tissue structures. Some of the current Irrigants and biocides follows in the next section.

### Toxic and Ineffective Dental Irrigants and Biocides

Chlorhexidine 0.12% is the most common irrigant for periodontal treatment. Alarmingly, Chlorhexidine is not indicated for use for periodontitis and is a poor irrigant in the periodontal pocket in that it is a large protein binding molecule. It is also poor at killing viruses and will not remove bacterial toxins such as methyl mercaptan. It also has a 2% incidence of hypersensitivity reactions in humans, including rare cases of anaphylactic shock. NaOCl is the most common irrigant used for debridement in endodontic procedures; NaOCl is also referred to as sodium hypochlorite, or bleach. In the treatment of gingivitis, NaOCl is almost never used to help debride and disinfect periodontal pockets.

When it is used, it is never used at a high enough strength to dissolve and remove necrotic tissue during root planning, aid in scaling plaque and calculus from tooth surfaces, or to aid in debridement. NaOCl effectively dissolves necrotic tissue in root canals at high concentration, but it is classified as a caustic soda and is toxic to humans. Hypochlorite solutions liberate toxic gases such as chlorine when acidified or heated. The reaction with ammonia or with substances that can generate ammonia can produce chloramines which are also toxic and have explosive potential. Consequences of using the sodium hypochlorite include accidentally spraying or splashing NaOCl into eyes causing erosion of and damage to eye tissues. There are several mishaps present in dental literature that describe root canal irrigation problems; the most common accidents arising during root canal irrigation concern damage of the patients' clothing. Since sodium hypochlorite is a common household bleaching agent, even small amounts may cause severe damage to clothing.

In certain endodontic procedures, rinsing of a root canal can result in the rinse entering the maxillary sinus, causing tissue destruction and allergic reactions. Saline rinsing of the sinus is subsequently indicated with the intent of removing the NaOCl. An all too frequent occurrence is the accidental splashing of NaOCl into the eyes of the doctor or patient, causing immediate pain, profuse watering, intense burning, and erythema, the possible loss of epithelial cells in the outer layer of the cornea, severe irritation, burns, and/or corrosion that may cause vision impairment and blurred vision. Further, the inhalation of vapors is irritating to the respiratory system, may cause throat pain and cough, severe respiratory tract irritation and pulmonary edema. Also, accidental splashing or spilling on the skin causes severe irritation and burns or dermatitis; thus, prolonged skin exposure may cause destruction of the dermis with impairment of the skin to regenerate at site of contact.

In the event that a patient accidentally ingests a high concentration thereof, this may cause injuries to liver, kidneys, central nervous system and gastrointestinal tract pain and inflammation, burns and perforation of the esophagus or stomach. Other effects of the ingestion may cause gastrointestinal irritation, nausea, vomiting and diarrhea, circulatory collapse, confusion, delirium and coma. Although concentrations high enough to cause these maladies are not typically used in endodontic or periodontal procedures, the use of NaOCl nevertheless carries all of these risks.

A 2010 study completed by Cobankara, Ozkan and Terlemez comparing organic tissue dissolution capacities of sodium hypochlorite and CD, it was concluded that when compared to NaOCl, CD does dissolve pulp tissue, however this is not supported by other studies. CD produces little or no trihalomethanes, a known animal carcinogen and a suspected human carcinogen. Other disadvantages of sodium hypochlorite are that it is unstable and that it disintegrates when heated. This also happens when sodium hypochlorite comes in contact with acids, sunlight, certain metals and poisonous and corrosive gasses, including chlorine gas.

Due to the presence of caustic soda in sodium hypochlorite, when used in water, the pH of the water is increased. When sodium hypochlorite is dissolved in water, two substances form, which play a role in oxidation and disinfection; these are hypochlorous acid (HOCl) and the less active hypochlorite ion (OCl—).

While these problems associated with use of NaOCl as a root canal irrigant sound extreme, dental literature documents more extreme dangers of its use, and references many cases where patients were forced to undergo facial surgery under general anesthesia to excise necrotic tissue caused by injected NaOCl, and many weeks of recovery before being able to resume the root canal procedure. Despite this long list of dangers, NaOCl remains the primary irrigant and disinfectant used in endodontic procedures, and despite the fact that the FDA has not specifically approved its use for such purpose, Chlorhexidine remains the most common irrigant for periodontal treatment.

### Irrigant Problem Summary

As is exhaustively described, and is well known to those skilled in the art, the current practice methods and standard of dental care employed to treat periodontal disease, or to perform endodontic procedures (a) employs the use of highly caustic irrigant solutions that can cause physical injury, hypersensitivity reactions and unintended tissue damage and discomfort to the patient and their clothes, (b) fails to fully remove bacterial toxins in hard-to-reach areas at a procedure site, and (c) often times are not adequate in fully disinfecting hard-to-reach deep gingival pockets or root canals of the teeth.

Some solutions to this problem have included the use of a device sold by Endo Technic, an expert manufacturer of endodontic hand pieces. This company makes and sells an integrated NaOCl delivery system, advertised specifically to allow endodontists to deliver NaOCl into the root canal for safer rinsing. The offering of Endo Technic's device also reinforces the recognition of the known dangers of use of NaOCl during oral procedures.

Method of Activating a Biocide with Acoustic or Photoacoustic Frequency

For years, sonic and ultrasonic devices have been used to clean items by pulsing acoustic wave energy through a fluid. Ultrasonic cleaning devices are routinely used in the jewelry industry to effectively clean the nooks and crannies of rings, watch bands and necklaces that would otherwise be unreachable using brushes or other mechanical cleaning devices. It would therefore stand to reason that sonic or ultrasonic devices could be used for oral care and indeed, they are.

Sonicare, Oral-B Pulsonic and other toothbrushes delivering sonic pulses have been sold commercially for some time. Further, in the professional oral care world, the Photon Induced Photoacoustic Streaming laser (“PIPS”), which is also marketed under the trademark Photo Hydro Acoustic Systems Technology (“PHAST”), represents an advancement in endodontic and periodontal treatment, although other sonic and ultrasonic producing devices have been used in the dental industry for years.

However, the article Lack of antimicrobial effect on periodontopathic bacteria by ultrasonic and sonic scalers in vitro. J Clin Periodontol. 2000; 27:116-119 by Schenk et al, reported that no statistically relevant reduction in periodontal pathogens resulted from up to 150 seconds exposure to various magnetostrictive ultrasonic scalers, sonic scalers, or ultrasonic cell disruptors typically used in dental procedures. While the devices were effective in debriding and removing scale and calculus during periodontal procedures, used intraorally, they have no clinically acceptable effect on killing bacteria. In contrast to these devices, some but not all studies on PIPs wave photoacoustic devices have shown ability to kill bacteria.

It should be understood that smear layers are created on dentinal tissues whenever root pulp is removed using hand or rotary instruments. This thin (1-2 microns) layer of denatured cutting debris creates a tenacious bond with the underlying tissue and, in fact, is often the surface to which restorative materials are cemented. Additionally, during pulp removal, cutting debris is forced variable distances into dentinal tubules. These “smear plugs”, along with the smear layer decrease dentin permeability, dentin sensitivity and surface wetness.

Research has shown the effectiveness of ultrasonic agitation during root canal therapy in helping to remove the smear layer. Further, ultrasonic agitation is sub-ablative, and can actually remove smear plugs from the dentinal tubes. However, foci of this research was simply to determine rinse and ultrasonic frequency combinations to break the bond between the smear layer and dentinal surface; this did not investigate the correlation between ultrasonic agitation and destruction of bacteria colonies established within the dentinal tubules, transverse root canals, anastomoses, tooth cracks, nor in abscess pockets that form near the root tips of infected roots.

For efficiency purposes and of this disclosure, references to ultrasonic should also be understood to mean sonic, sonication, photoacoustic, or any other acoustic frequency-producing device that would be used to agitate a rinse solution, and used in the treatment of periodontal disease or root canals. The advantage of using ultrasonic frequency in treating periodontal disease, or in conjunction with performing endodontic procedures, is that the excitation of a fluid by acoustic waves helps break bonds between biofilm and tooth surfaces, and helps in debridement of necrotic and diseased tissue, and along with it, bacteria.

Although the application of ultrasonic devices during dental procedures is well known, the use of acoustic delivered laser energy is much less known in dentistry. In most all cases, the fluid through which the frequencies are carried is sterile rinse water. Some ultrasonic devices incorporate a means to deliver a fluid stream, as well as suction to remove the rinse solution, debrided tissue and bacteria. These ultrasonic frequencies produce heat that can cause patient discomfort; excessive heat generation is not desired in dental procedures.

The PIPS laser previously mentioned does create some heat. However, PIPS prevents undue heat build-up problem by pulsing waves in its frequency, thereby creating intermittent frequency bursts that produce almost no heat on human tissues, in a sub-ablative process. It should be understood that the primary reason that heat production is retarded is to reduce patient discomfort, and manufacturers addressing heat production in thermal lasers, sonic and ultrasonic devises advertise lower heat production as a feature intended for patient comfort. PIPS technology used in Erbium YAG lasers with a wave length of 2940 nm at settings used to debride root canals and gingival pockets produce less than 1% rise in temperature and are therefore of no concern for heat production.

FIG. 1 is an exemplary diagram showing the anatomy of a healthy human tooth. In the drawing, a tooth crown (1) is shown comprising an enamel surface (4) encapsulating semi-hard and porous dentin (5). Further encapsulated within the dentin, a root pulp (6) is shown. Nerves and blood vessels surrounded by dentin and integral to the root pulp comprise the root (2) of the tooth. The root canal vessels delivers nutrients to the tooth through tubules in the porous dentinal walls. As the roots extend from the crown portion towards the root tips, the thickness of the dentinal walls diminish, thereby allowing one or more smaller roots, referred to as transverse canals (3) to create their own pathway through the dentin, most often at sharp angles to the primary root axis.

The tooth is attached to the jawbone, more accurately referred to as alveolar bone (7) by a combination of the periodontal ligament (9) and cementum (8). The tooth nerves and blood supply leave the root through the apical foramen 10 and ultimately connect to the primary maxillofacial veins, arteries and nerves 11. The top of the gums, also referred to as the gingival crest 12 is shown tightly located against the tooth approximately where the enamel crown terminates at the dentin. Abscesses may occur in the soft tissue of the root, semi-hard tissue of the dentin, soft tissue surrounding the hard structures of the teeth, at or adjacent to the apical foramen, within the maxillofacial blood vessels and verves, or within the alveolar bone.

FIG. 2 is an exemplary illustration of a human tooth exhibiting the effects of periodontal disease. In the drawing, a representative tooth is illustrated showing that the gingival crest 21 had receded and pulled away from the tooth surface, and more specifically, has receded from the point at which the gingival crest contacts the tooth near the termination of the edge of the crown. As can be readily seen, a roughly surfaced, highly porous calculus 20 has formed against the tooth surface. Also commonly referred to as tartar, calculus forms when the bacterial colonies that comprise plaque, a biofilm, are allowed to form and grow over a period of time. Calculus forms above, as well as below the gum line as shown in the continuation of the calculus to form sub-gingival calculus 23.

As the bacteria pockets continue to grow, they and their bacterial toxins create an inflammatory reaction, resulting in the infiltration of inflammation 22 into underlying gingival tissue. As periodontal disease progresses, the alveolar bone begins to recede 24, and along with it, there is a loss of epithelial attachment 25 between the bone and healthy tooth structure. Disease progression results in edema, infection, and degeneration of the periodontal ligament and cementum 26. Deep periodontal pockets 27 subsequently harbor bacterial colonies and volatile sulfur compound toxins that perpetuate the disease until the ultimate loss of bone and tooth. Root abscesses result when bacteria enter the root pulp or root, either through dentinal tubes, apical foramen, transverse canals, tooth cracks or other entry points.

FIG. 4 is a prior art exemplary illustration showing multiple magnified sections and details of the dentin 82 and dentin tubules 85 of a human tooth. In the illustrative drawing, a cross section of a tooth with two root canals is shown 80. An enamel crown 81 overlays dentin 82. Because dentinal tubules are merely 1 μm to 2 μm in diameter, they are not shown in this section of the tooth illustration. Alveolar bone 83 is shown on both sides of the tooth, but in fact, alveolar bone typically surrounds the tooth.

The magnified view 84 is a representative illustration of a plurality of tubules 85 penetrating transversely through the thickness of the dentin 82. These dentinal tubules deliver micronutrients to the dentin from the root pulp. Arteries, veins and nerves, and odontoblasts 86 connected to the nervous and circulatory system within the pulp, supply nutrient fluids from the root pulp into the tubules. Dentinal tubules, when clear, communicate not only fluids between the inner and outer tooth surfaces, but they can also provide a bi-directional path for transmitting bacteria. As a consequence, billions of bacteria originating from periodontal disease inflammation may penetrate into the tooth root pulp and root canals, and conversely, inflammation within the root canal, caused by cracked teeth, dental carries or other trauma may be communicated to the gingival tissue.

Because of the inherent problems with the related art, there is a need for a new and improved oral care system and method for the treatment of certain oral pathological conditions that require the cleaning and cleansing of the underlying scaffolding of the tooth.

### BRIEF SUMMARY OF THE INVENTION

The invention generally relates to an oral care system which includes various embodiments hereinafter described, including a method of cleaning a tooth comprising steps of preparing a tooth for irrigation, applying chlorine dioxide to an affected region of the tooth and activating the chlorine dioxide using a tool.

The first embodiment of the novel invention taught herein envisions a dental professional performs a method of cleaning a tooth having the following steps. A root canal procedure is performed thereby preparing the tooth such that an orifice is created into the affected regions of the tooth. All of this is so that the tooth can be effectively irrigated. Chlorine dioxide is added to the affected region of the tooth that is subsequently treated with an actuating tool, typically a laser or similar device, thereby releasing its cleansing properties. It should be understood that in preparing the tooth either a rubber dam is applied around the tooth or the tool is directly in contact with the sulcus of the tooth. The rubber dam is sealed around the tooth with a liquid dam material whilst ensuring that it is not sealed against the tooth structure. In applying the chlorine dioxide it should be clear that it is filled with chlorine dioxide thereby submerging all free gingival margins.

A sanitizing process for a diseased tooth having a dental surface is performed by a dental professional opening the surface for access to an affected region therein. He or she then removes tissue from the region and fills the region with chlorine dioxide acting as a cleanser. The chlorine dioxide cleaning action is effected through the use of an agitation tool that agitates the substance thereby causing it to cleanse the affected region. It should be understood that the tool is alternatively a sonication type, a laser agitation tool or similar device. To assist in clearing out the region pure water is added thereto and in hopes of finishing off the cleanser the agitation tool is used proximate the affected region.

Next a dental professional places a tissue dissolving liquid in the region and activates an agitation tool proximate the affected region. This part of the process is repeated another two times. That is, the placing a tissue dissolving liquid in the region and activating an agitation tool proximate the affected region are repeated two more times. Then pure water is added to the region followed by activation of an agitation tool proximate the affected region; next a cleanser is added to the region and an agitation tool is used to agitate the liquid proximate the affected region. The process continues with a user adding pure water to the region and a subsequent activation of an agitation tool proximate the affected region. Next, a dental professional adds another cleanser to the region and uses an agitation tool proximate the affected region. It should be understood that another cleanser is chlorine dioxide. Pure water is added to the region after which an agitation tool is activated proximate the affected region whereupon the region is dried. At the end of the process a dentist or other professional fills the affected region of the tooth and closes an access hole.

In a third embodiment, a dental professional prepares a root canal in a diseased tooth for therapeutic treatment. First a chamber is opened within the tooth to create the root canal and tissue is removed therefrom. Chlorine Dioxide is added to the chamber for cleansing the interior thereof and a tool is used to actuate the cleansing properties of the chlorine dioxide. Afterwards pure water is added to the chamber.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an exemplary prior art diagram showing the anatomy of a healthy human tooth.

FIG. 2 is an exemplary prior art illustration of a human tooth exhibiting the effects of periodontal disease.

FIG. 3 is an exemplary illustration of a sequence of steps and the associated methods of performing root canal therapy. FIG. 3A illustrates a healthy tooth. FIG. 3B shows a damaged or infected tooth. FIG. 3C shows a tooth having pulp become inflamed FIG. 3D shows the start of root canal therapy by opening the crown FIG. 3E shows part of the root canal procedure whereby pulp is removed. FIG. 3F shows the addition of a dam material around the perimeter of the tooth. FIG. 3G shows the use of a tool in the tooth. FIG. 3H illustrates the addition of a tissue dissolving liquid. FIG. 3I shows the repetition of a tool activation near the tooth. FIG. 3J shows the addition of a tissue dissolving liquid.

FIG. 3K shows the third use of a tool in the tooth. FIG. 3L illustrates the flushing with sterile water. FIG. 3M illustrates the use of a tool in the tooth chamber. FIG. 3N shows the insertion of a liquid into the tooth. FIG. 30 shows the usage of a tool in the tooth FIG. 3P shows the irrigation using sterile water. FIG. 3Q illustrates the use of a tool in the tooth. FIG. 3R shows the drying of each of the canals. FIG. 3S illustrates the obturation of the tooth. FIG. 3T shows how the dentist fills the upper cavity and finalizes closure thereof.

FIG. 4 is a prior art exemplary illustration showing multiple magnified sections and details of the dentin and cementum of a human tooth.

FIG. 5 is an exemplary illustration of the therapeutic processes used to treat periodontal disease.

FIG. 6 is an exemplary illustration showing electron microscope photographs of a smear layer and smear plugs on the dentinal surface of a human tooth, tubules containing microbial toxins and bacteria, and the dentin with clear tubules after removal of the smear layer.

FIG. 7 is an exemplary table of comparative laboratory text results, and a corresponding graphical representation of the average reduction in Volatile Organic Compounds of each compound listed in the table.

FIG. 8 is an exemplary table of comparative laboratory test results illustrating Minimum Inhibitor Concentration of CD at a concentration of 37 parts per million versus Chlorhexidine (CHX) in effectiveness to destroy bacteria and yeast.

FIG. 9a-9c is an alternative exemplary illustration of a sequence of steps and the associated methods of performing root canal therapy to that found in FIG. 3.

FIG. 10 is an alternative exemplary illustration of the therapeutic processes used to treat periodontal disease to that found in FIG. 5.

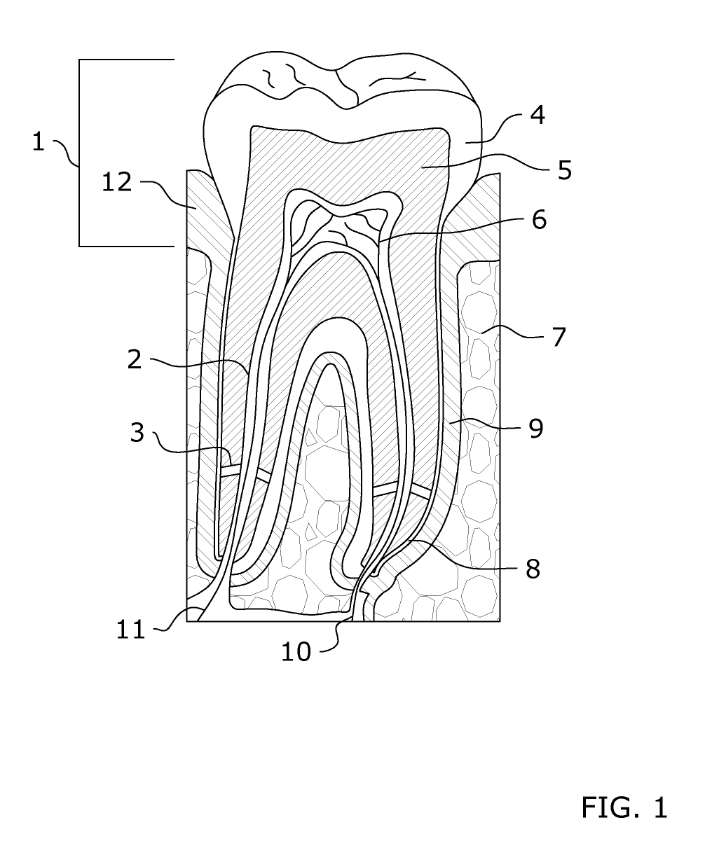

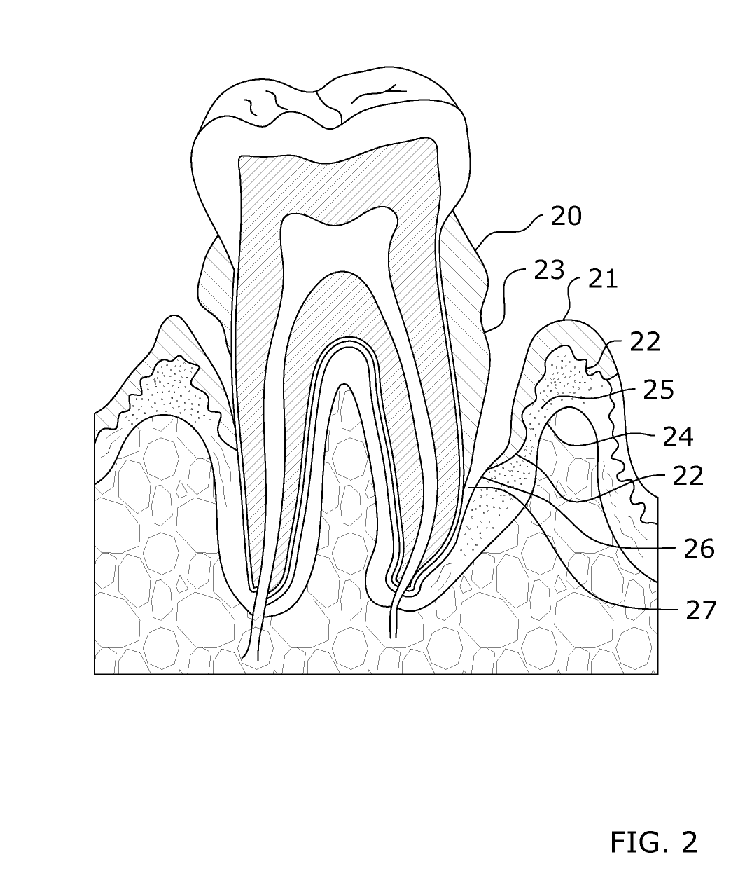

### DETAILED DESCRIPTION OF THE INVENTION

### A. Chlorine Dioxide (CD) not Anticipated in Endodontic Procedures.

As stated previously, for the purposes of this disclosure, it should be understood that chlorine dioxide, its chemical formula ClO₂, the liquid form of chlorine dioxide, the gel form of chlorine dioxide and the abbreviation “CD” wherever referenced may be used interchangeably, and have the same meaning and chemical structure. Use of CD in specific endodontic treatments is not anticipated by the use of other disinfecting solutions. For example, the Endo Technic system previously described does not use CD. At the very least, a profit-driven enterprise that manufactures a product that's intended to deliver a disinfecting solution into the root canal would reasonably disclose and capitalize on every possible solution that would be used in order to appeal to a wider customer base.

### B. CD is a Preferred Oral Disinfecting Solution.

CD and chlorine share ‘chlorine’ in their names and emit similar odors, but they are fundamentally different chemical compounds, and generate radically different byproducts. CD acts by oxidation whilst Chlorine will combine to produce harmful by products, many of which are recognized as human carcinogens. CD will not hydrolyze to form acid, and is therefore less corrosive. Chlorine bleach is pH dependent and effective as a biocide at pH levels near 12, but CD is effective at all pH's below 12. Further, Chlorine bleach will not remove biofilm, but CD does. CD is available in two forms, active, and stabilized. It is illegal to transport active CD in large containers since it is an explosive; because of this, transported CD is always of the stabilized variety. This type, stabilized CD, is sodium chlorite and does not have the bacterial, fungal and viral killing ability of CD; however it does have a weaker oxidative potential and can remove some volatile sulfur compounds.

Next, CD is a gas and can remain dissolved in water for a short period of time as a chlorous acid/chlorine dioxide state before gassing out of the liquid. Further, while chlorine bleach is effective in destroying some bacteria, it is ineffective on cysts and protozoa. On the other hand, CD is a broad spectrum kill agent, effective in destroying aerobic, non-aerobic, gram-positive, gram-negative bacteria, viruses, fungi, spores, cysts and protozoa. It also destroys spore forming and non-spore forming pathogenic and saprophytic bacteria, and bacterial spores, one of the most resistant forms of bacterial life to disinfection.

CD is also effective against molds and yeast, and is extremely effective against acid tolerant bacteria such as E. Coli, and all the periodontal pathogens, including Porphyromonas gingivalis, Aggregatibacter actinomycetemcomitans, as well as the root canal pathogens including Enterococcus faecalis. It is effective against algae and protozoans including Cryptosporidium, Microsporiclium, and Giardia lamblia. Further, the viricidal activity of CD may actually exceed its bactericidal potency.

Studies have shown CD as being a potent antimicrobial agent, with a 5 log reduction (99.999%) within 30-60 seconds against Listeria, E. Coli, Pseudomonas, Salmonella, Staphylococcus, Streptococcus and others. However, since the primary chemical reaction of CD is oxidation, active CD is more effective as a biocide and viricide than stabilized CD or sodium hypochlorite.

An important consideration underlying the discovery of the present invention, with concentrations of CD from 25 to approximately 250 parts per million concentrations in aqueous solution, is the duration of time required to kill the targeted bacteria, virus or fungi. More specifically, microbes generally have two types of cell structures, prokaryotic and eukaryotic. Most bacteria are of the more simplistic type, prokaryotic, which have enzymes located immediately inside of the cellular membrane. Because of this, the oxidative attack of CD on these cells is nearly immediate.

On the other hand, fungi and protozoa are of the eukaryotic type where enzymes are located deeper in the cell structure; also, bacterial spores have many layers of protective material surrounding their enzymes, and are more resistant to oxidative attack. Therefore, the time that the bacteria, virus or fungi is exposed to a biocide or viricide is directly related to the effectiveness of the solution as a disinfectant.

C. Biocide or Viricide Use with Sonic, Ultrasonic or Photoacoustic.

Further, research failed to find any published information that specifically references the use of an oral disinfectant used in conjunction with sonic, ultrasonic or other photoacoustic device for any purpose, nor did any research anticipate the use of a biocide or viricide in conjunction with a sonic, ultrasonic or photoacoustic device.

In fact, applying an acoustic wave-generating device to periodontal or endodontic rinse solutions would be contraindicated because of the elevation of risk of injury to patient and caregiver. As would be known to those skilled in the art, the application of sonic excitation to the most commonly used oral disinfectant, NaOCl, would increase the danger and liability by creating toxic aerosols and splashes that would increase the chance of inhalation or contamination of the skin or eyes of both the patient and dental practitioner, and would therefore never be seriously considered as a viable or practical modality for endodontic procedures, nor to treat for periodontal disease.

It should be noted that in root canal therapy, there exists a theoretically perfect therapy, one in which 100% of the pulp, tissue, odontoblasts, smear layer, blood vessels, nerves, bacteria, fungi, viruses, and microbial toxins are removed from the tooth cavity, root canals, transverse canals, and the innumerable dentinal tubules. Given the state of the art in dental procedures, processes, materials and devices, theoretical perfection is currently out of reach. As will be shown, the improved methods and processes described herein step closer to the perfection milestone, and are a demonstrable advancement over the current state of the art.

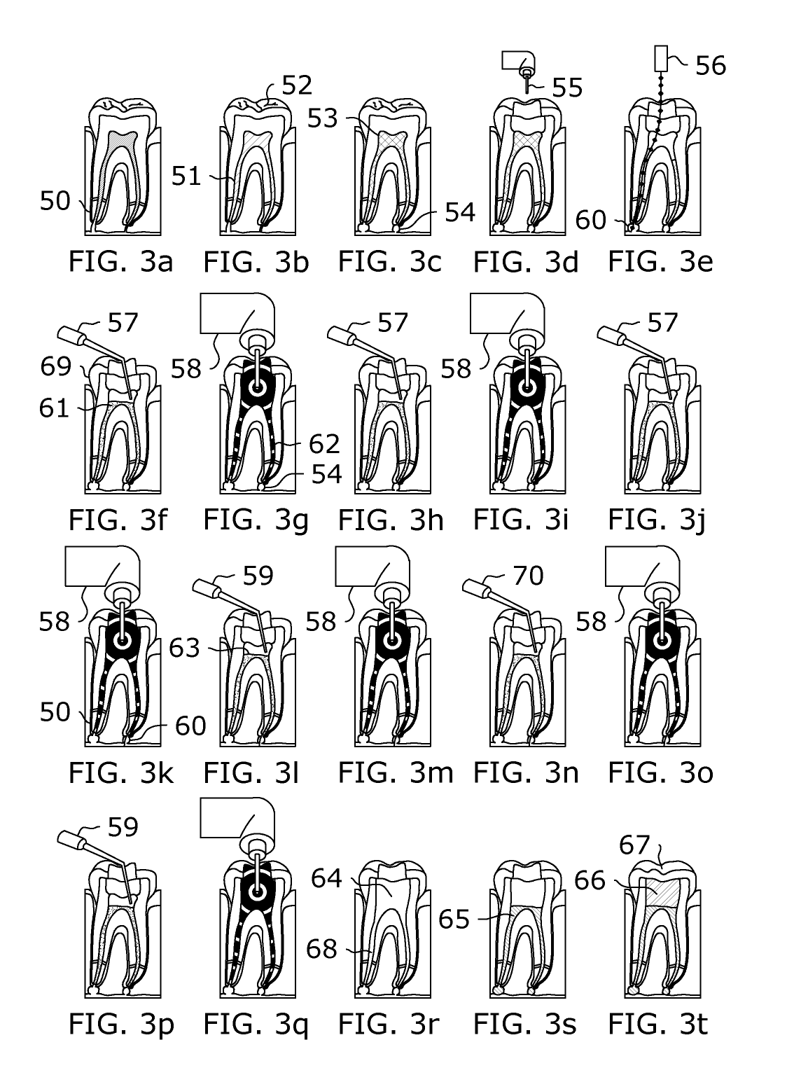

FIG. 3 is an exemplary illustration of a sequence of steps and the associated methods of performing root canal therapy as described in an embodiment herein. It should be clearly noted that the process of root canal therapy as illustrated by Encyclopedia Britannica fails to reference any of the illustrated therapeutic processes beginning at canal irrigation 61, through the final fluid evacuation step 47 preceding obturation, completely ignoring the clinically proven importance of debridement, irrigation, detoxification and the disinfecting process. By text reference only, the process states that: “water or sodium hypochlorite is used periodically to flush away the debris.”

Similarly, the American Dental Association (ADA), and by reference to the ADA process, the Mayo Clinic, follow the same procedure taught by the Encyclopedia Britannica. Discussion disregards the paramount importance of bacterial and toxin disinfecting, and these resources are moot regarding negative health issues associated with the practice of disinfecting with NaOCl. Neither chlorhexidine nor NaOCL remove bacterial toxins that, in fact, produce inflammation and disease.

In stark contrast, the improved root canal therapy method centers on a novel and previously unknown method of destroying dangerous oral microbial toxins; this provides a statistically improved and important method and process of improving clinical outcomes from endodontic and periodontal procedures. For purposes of efficiency, reference to illustrations in the following sequence all refer to cut away sections of the tooth, illustrating the therapeutic processes performed within the tooth cavity.

The improved system and method of the present invention employs the following mechanism that ensures that the sodium chlorite and active chlorine dioxide of the present invention most effectively removes bacterial toxins:

The reaction of sodium chlorite (stabilized chlorine dioxide) with hydrogen sulfide.

H²S+2NaClO^{2□}2NaCl+SO⁴═+H²

One mechanism by which the “Stabilized Chlorine Dioxide” containing mouthrinse products are purported to eliminate halitosis is by the oxidation of the odoriferous volatile sulfur compounds into non-volatile, non-odoriferous molecules such as the corresponding sulfates, sulfonates and sulfones.

The reaction of hydrogen sulfide, expressed as the sulfide ion, with active chlorine dioxide which has been activated from the stabilized form is:

S⁻²+CLO₂□SO₄⁻², sulfate ion

The reaction of methyl mercaptan with active chlorine dioxide is:

CH₃SH+CLO₂□CH₃SO₂OH, methyl sulfonic acid

It should be further noted that effective sonication employed in the following process and sequence relies on proper device set up prior to the procedure. For instance, the settings for the Er:YAG laser, also commonly known as the PIPS method of applying photoacoustic shock waves to fluid within a tooth cavity, should be:

Frequency: 15 Hz

Power: 15-20 mJ

Pulse duration: 50 microseconds

Air:Water ratio: 0:0

While these settings are presented for a PIPS technique, they are not intended to be limiting, and use of alternative sonic or ultrasonic devices will correspondingly require different setups in order to deliver an equivalently effective sonic agitation of the fluid within a root canal and tooth cavity.

A healthy tooth 30 is shown FIG. 3A with the representative anatomy discussed previously. In particular, healthy transverse canals 50 are also illustrated. An infected root 51 FIG. 3B is shown in the illustration, with a tooth crack 52 shown as one of the many possible causes or sources of the infection. As the infection progresses in the tooth 33, the root and pulp become inflamed 53 FIG. 3C. In many cases, an abscess 54 forms in or around the root structure as a response to the inflammation. The diagnosis of such a condition, using x-rays, visual evidence and stated patient symptoms, most often result in a prescribed root canal therapy.

Root canal therapy begins FIG. 3D by opening the crown 55 of the inflamed tooth 33, a procedure typically performed by the dentist using a rotary drill, flaring the edges wider at the crown surface. In a root canal, this process fully exposes the pulp and root structure of the tooth.

The opened tooth 34 is ready for the next process, removal of the root pulp. This is accomplished with the use of hand-held files or rotary files. Using relatively large endodontic files 56 FIG. 3E, the portion of the root and pulp closest to the crown is first removed. Using files of decreasing diameter, the deeper, small diameter roots pulps are similarly removed, sometimes with the benefit of removing the causes of abscesses that form in the area of the apical foramen 60. The dentist continues filing the canals to a smaller width than their usual protocol with either hand files or rotary files. Not shown, however hand files, rotary files and pathfinder rotary files, should be used with EDTA or other types of lubricant pastes, taking care not to go past the apex. Irrigation with a CD rinse should be done frequently.

Upon removal of all of the pulp tissue from the tooth canals 35, a dental product sold commercially under the name of Fast Dam 69, or an equivalent barrier material is installed FIG. 3F around the perimeter of the tooth to retain irrigation fluid at a fluid level equal to or above the top of the drilled crown, and to prevent seeping or spilling of irrigants or debris into the oral cavity. The opened canal is then filled with a bleach solution or alternative tissue dissolving liquid 57.

Insert (FIG. 3G) the laser PIPs tip of the Erbium YAG laser hand piece 58 into center of the tooth filled with the 3% to 6% sodium hypochlorite bleach solution or alternative tissue dissolving liquid 36, and activate the hand piece to deliver the acoustic pulses into the bleach solution for about 30 seconds in the tooth chamber only and not into the canals and not touching the tooth. If an ultrasonic or sonic instrument is used, the sonic or ultrasonic files or tips should be placed into each canal, touching the canal surfaces consistent with the intended design. The sonication will agitate the bleach in the deep and narrow portions of the root canal 62, delivering the beneficial effect of removing debris, and loosening and removing root canal tissue. After PIPS, pause for 30 seconds or more to allow adequate time for the bleach or alternative tissue dissolving liquid to dissolve tissue and to kill bacteria within the canal, and in the dentinal tubules in which the bleach is in communication with.

After the pause period of 30 seconds, add (FIG. 3H) additional bleach or alternative tissue dissolving liquid 57 to the tooth cavity 37 to replace any liquid displaced by the procedure. In the next step 38, repeat the sonication procedure (FIG. 3I) using the hand piece 58 for a second time, using the same time-based guidelines as previously specified. Prior to continuing to the second phase 37 of replenishing the bleach or alternative tissue dissolving liquid, pause for 30 seconds or more to allow adequate time for the liquid to kill the bacteria and dissolve tissue within the canal, and in the dentinal tubules in which it is in communication with.

It should be noted that during all times when active sonication is being conducted, a dental assistant should ensure that the root canal chamber is never emptied of bleach or alternative tissue dissolving liquid, and should replenish as needed to maintain adequate fluid levels, otherwise the PIPS photo acoustics will not work well at bringing those pulses to all anastomoses and tubules in the canal.

Add additional bleach or alternative tissue dissolving liquid 57 to replenish the irrigation level as shown in the illustration 39 (FIG. 3J), and for a third time, as shown in the illustration 40, re-insert (FIG. 3K) the sonic or ultrasonic files or tips into each canal, touching the canal surfaces consistent with the intended design, or use the PIPS tip in the chamber only to maximize agitation of the liquid, debridement, and the loosening and removal of the canal soft tissue.

It should be noted that any difficult-to-reach anastomoses 60, as well as transverse root canals 50 that are in fluid communication with the bleach or alternative tissue dissolving liquid irrigant in the root canal chamber, will have been maximally agitated, chemically disinfected, detoxified, and mechanically debrided; thus, this helps to minimize the possibility of re-infection that results from inadequate disinfecting, detoxification, and debridement of processes presently considered as the dental standard of care.

The next step 41 is to flush (FIG. 3L) the bleach or alternative tissue dissolving liquid from the canals by irrigating with sterile water 59. Irrigate each canal fully. When sterile water has replaced the bleach or alternative tissue dissolving liquid in the canals, 42, repeat (FIG. 3M) sonication in each canal for about 30 seconds or PIPs the chamber for 30 seconds. After sonication, advance to the next step 43 and irrigate each canal with ETDA, (FIG. 3N) ensuring that the sterile water is fully displaced by ETDA. This step can have EDTA replaced by CD.

The tooth 44 with the canals fully irrigated with ETDA or CD then undergoes (FIG. 30) additional sonication for about 30 seconds in each canal, depending on the number of roots and complexity of root canal structure or PIPS activation in the chamber only for 30 seconds. If EDTA was used, the next irrigation step 45, use sterile water, and irrigate (FIG. 3P) for about 30 seconds. If EDTA was used, follow the water irrigation with irrigation of CD for 30 seconds. In the final sonication step 46, the PIPS or other sonic hand piece is delivered (FIG. 3Q) back into the sterile water within objective of removing any remaining ETDA or CD. Sonicate for about 30 seconds.

To ready the tooth 47 for obturation, 65, dry each of the canals (FIG. 3R), but do not dessicate them. It should be noted that the main cavity of the tooth 64 and the canals 68 will also be dried in the process. Obturation of the tooth 48 is initiated (FIG. 3S) by filling the canals with a commercially available product such as gutta percha and sealer or sealer only 65. The final phase 49 requires the dentist to then fill the upper cavity 66 (FIG. 3T) with a commercially available product such as composite or other acceptable obturation system, and may finalize closure by installing an artificial crown 67.

### D. Final Considerations on FIG. 3.

In the previous sequence and method of the present invention, reference is made to a specific time duration for sonication of bleach or alternative tissue dissolving liquid, CD, ETDA and sterile water, however, these time durations are not meant to be limiting. More or less time will be adequate for thorough disinfecting, detoxification, removal of the dentinal smear layer, sub-ablative removal of smear layer and bacteria from dentinal tubes, and debridement depending on various factors including chemical concentrations, sonic frequency, or root depth. The duration of sonication is intended to serve as guidance to the practitioner, and should not be considered an absolute minimum or maximum time to adequately disinfect the root canals.

It should also be noted that the obturation system and crown as illustrated 48 may be temporarily installed in instances where the dentist desires a period of time between initial root canal therapy and obturation to observe for any re-infection prior to permanent restoration, or if the dentist is confident in the just-performed therapy in regards to disinfection and debridement, may install a permanent filing and crown, completing the therapy and repair. It should be further noted that the dentist may modify these steps according the conditions that guide his or her professional judgment in patient care.

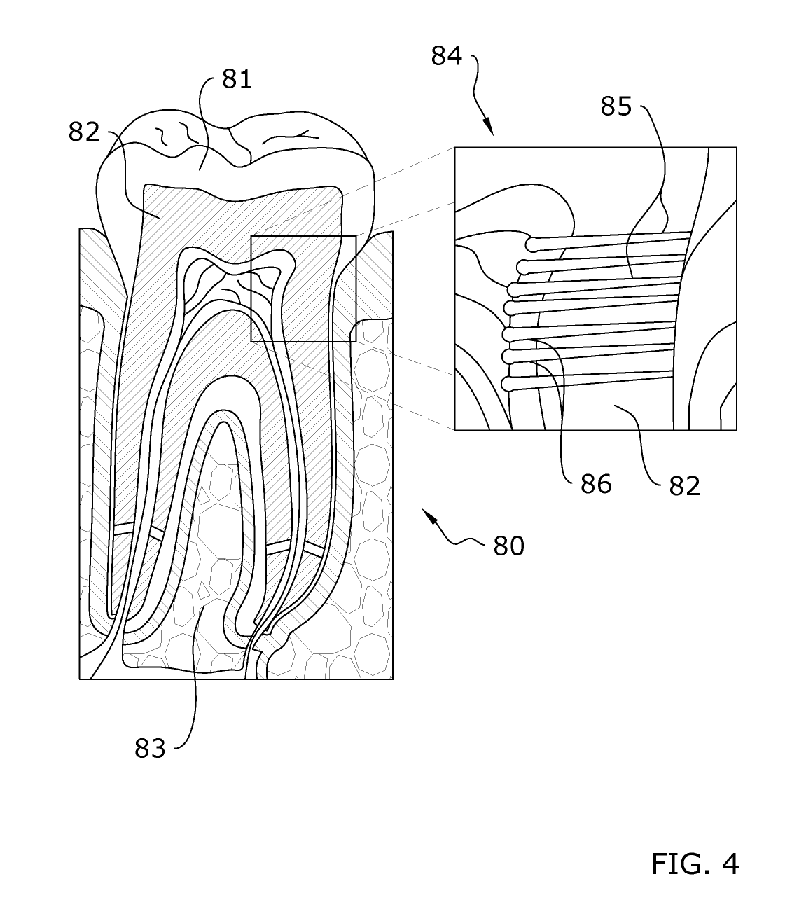

Returning to FIG. 4 it should be understood that with regard to periodontal procedures, it is generally the objective to remove scale and debris that form over the cementum that obstructs the tubules, and to remove sub-ablative debris from the surface of the cementum. In the present invention, a new and improved process of periodontal therapy is taught whereby an ultrasonic scaler, magnetostrictive scaler, piezo scaler, or PIPs laser is used in combination with CD to accelerate activation of active CD, and provide laser acoustic, sonic or ultrasonic shock waves within the CD to more effectively remove the biofilm, VSC toxins, and or debris from the root and enamel surfaces of the teeth being treated, and further to loosen and remove sub-surface debris from cementum. This allows for more effective destruction of microbial toxins.

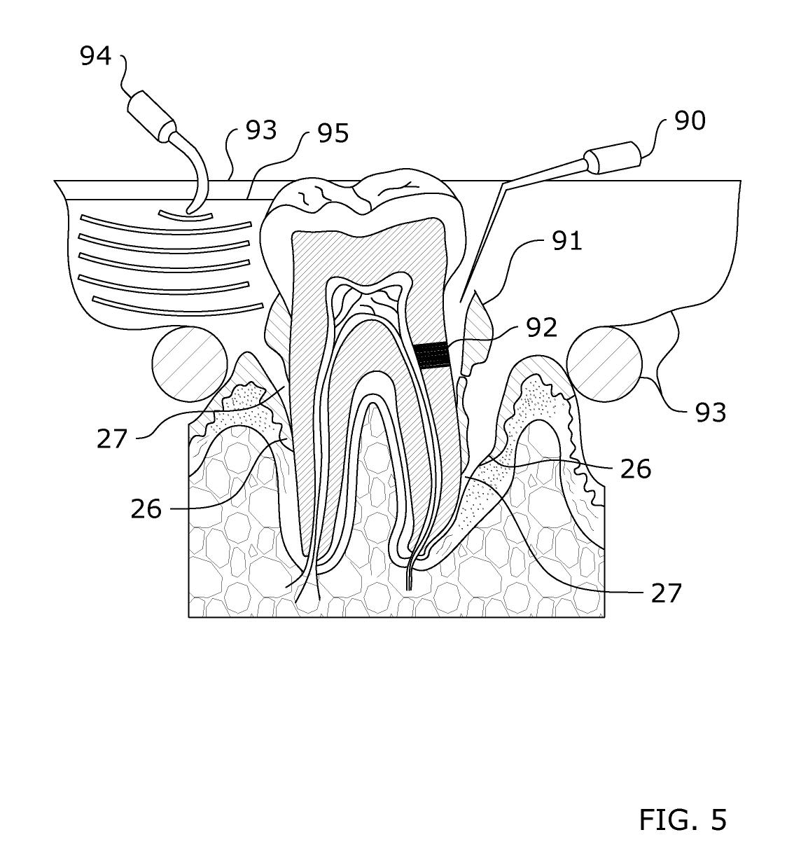

FIG. 5 is an exemplary illustration of the therapeutic processes used to treat periodontal disease, illustrating a cross-sectional view of a tooth, gingival and bone structure, and the perimeter structure of a dental reservoir. An ultrasonic scaler, magnetostrictive scaler, piezo scaler, laser, or hand scaler 90 is used to remove the calculus 91 from the root and enamel surfaces of the teeth being treated. One objective is to expose the cementum 92 in order to allow for sub-ablative removal of oral microbial toxins and debris from the cementum. A rubber dam barrier reservoir may be used and 93 is sealed around the tooth or a quadrant of teeth with exposed gingival margins and interdental gingival papillae, thereby allowing for the maintenance of a minimum fluid level of CD 95 to fill the gingival pocket areas 26, 27 surrounding the tooth, but preferably, the rubber dam should allow the fluid level of the CD to be maintained even deeper, rising to the cusp or the crown.

It should be noted that flooding with the CD irrigant is initiated before, and continued during and after the following detailed process. The CD is delivered within the dammed reservoir either by providing continuous irrigation by manual means such as an irrigation syringe, or by a sonic, laser or ultrasonic hand piece capable of delivering a continuous flow of CD. The tip of a sonic, laser or ultrasonic hand piece 94 is inserted into the periodontal pocket areas that are flooded by CD, and activated. A PIPs laser may not be inserted into the periodontal sulcus or pocket but instead submerged into the CD fluid just outside the margin of the gingiva if previous calculus removal has been done.

This sonication accelerates the creation of active CD gas, and agitates the CD to aid in debridement and removal of biofilm and microbial toxins from surrounding tissue and tooth surfaces. The duration of this sonication should be sustained for a period of about 30-50 seconds in the treatment area around each tooth, depending on the degree of advancement of the periodontal disease, periodontal pocket depth, the number of periodontal pockets, skill level of the practitioner, or other factors suggesting longer sonication cycles as determined by the dentist. Following initial sonication, pause for about 30 seconds to allow active CD to destroy contacted bacteria, fungi, viruses and toxins.

After the 30 second pause, repeat the entire sequence just described two additional times. It should be noted that in some cases, repeating the just-described sequence for a total of two times has been shown to effectively deactivate microbial toxins, kill pathogenic microbes and debride the area of necrotic tissue and debris, obviating the need for a third cycle. Finally, remove reservoir dam. Thereafter, release the patient with post-operative care instructions that include in-home use of CD rinse.

It should be understood that FIG. 5 is showing a tooth with a rubber dam fitting tightly around the tooth onto the gum tissue and it forms a reservoir cupped up and away from the top of the tooth. This is to hold the CD rinse in a pool around the crown of the tooth and also allows the liquid to completely bathe the tooth and the opening of the gingival sulcus. Then the sonicator or PIPS laser can be put into the liquid bath and the photo acoustic sound waves with drive the CD down into the disease periodontal pocket to kill bacteria, fungi and viruses; thus is shown a way of holding the liquid abound the tooth to make PIPS work because the laser tip works best if submerged in the liquid.

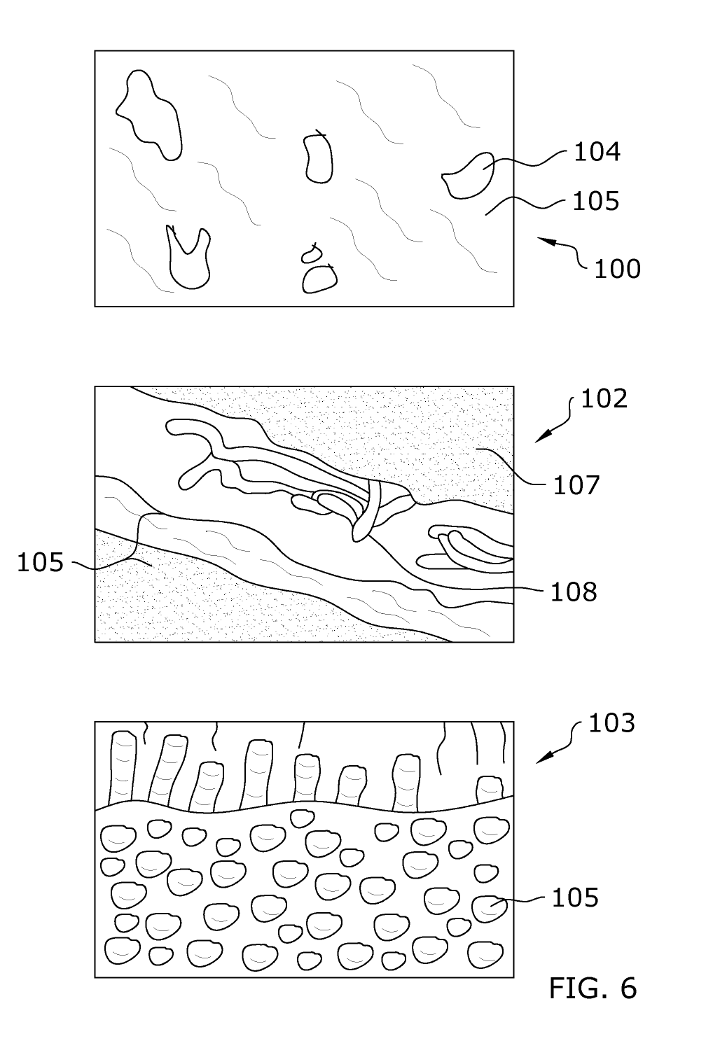

FIG. 6 is an exemplary illustration showing electron microscope photographs of a smear layer and smear plugs on the dentinal surface of a human tooth, tubules containing microbial toxins, and the dentin with clear tubules after removal of the smear layer. The diagrams demonstrate the critical importance of removing surface and sub-surface debris from the dentinal walls prior to root canal obturation.

In the upper photo 100, the dentinal surface of a root canal is shown with a thick layer of debris 105, referred to as a smear layer. The raised, relatively roughened surfaces 104 appearing in the field of the smear layer are disruptions caused by the underlying but obstructed dentinal tubules. Obturation of the root canal and tooth opening prior to full removal and disinfection of the smear layer often results in trapping microbial toxins under the smear layer, and within the dentinal tubes, thereby allowing bacteria to remain to cause re-infection. Failure to remove microbial toxins and bacteria during root canal therapy is a primary cause of post-operative inflammation and re-infection, often requiring additional therapy.

In the center photograph 102, a sectional view through two dentinal tubules 106 traversing through the dentin 107 are shown. The upper tubule is host to a colony of bacteria. As can be readily determined, the bacteria are not located on the dentinal surface within a root canal, but rather projecting not less than 12 μm into the dentin. One of the most critically important steps in root canal therapy is to destroy microbial toxins and bacteria below the dentinal surface, and especially into the roughly 2 μm diameter tubules.

While there are practical limits to a dentist's ability to excavate bacteria and debris from the thousands of minute dentinal tubules using state of the art mechanical devices, the system and method of the present invention has been demonstrably shown to remove debris, bacteria and destroy microbial toxins below the dentinal surface.

In the lower photo 103, it is readily noticeable that after applying the system and method of the present invention, the smear layer has been removed, and the dentinal tubules 105 have been reopened. The sonication of the CD rinse within the root canal has effectively removed the smear layer by mechanical, acoustic and chemical means, and the sonic or ultrasonic acceleration of the active CD reaction has effectively neutralized toxins and bacteria on the dentinal surface, as well as sub-ablatively, and into the dentinal tubules. The method of the present invention that combines the use of a sonic or ultrasonic device with active CD results in a clinically improved therapy and prognosis when compared to the methods practiced under the current dental standard of care.

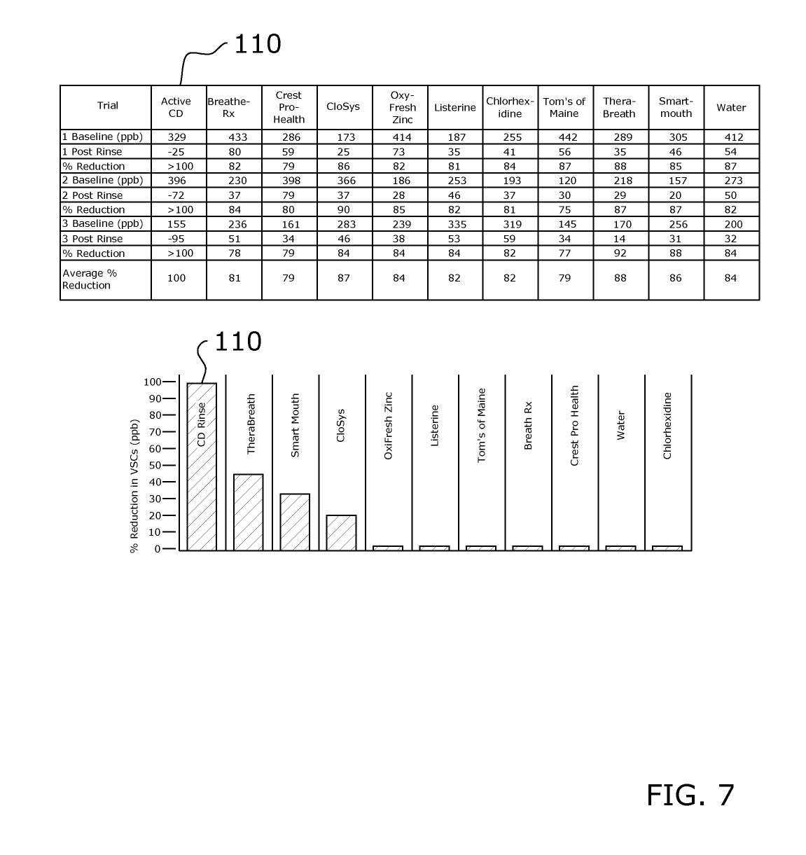

FIG. 7 is an exemplary table of comparative laboratory text results, and a corresponding graphical representation of the average reduction in Volatile Sulfur Compounds of each compound listed in the table. More specifically, the table data and corresponding chart presents the results of controlled laboratory tests conducted on Aug. 11, 2012. The in-vitro tests simulated actual patient-use conditions anticipated in root canal and periodontal procedures. The measurements compare oral rinses based on their ability to destroy or reduce microbial VSC toxins after a 30 second exposure. The tests were completed for a variety of toxins including those produced by Porphyromonas gingivalis, Prevotella intermedia, Tannerella forsythia, Treponema socranskii, and Streptococcus mitis, all of which are routinely present in the oral cavity during periodontal and endodontic procedures.

As can readily be seen, the active CD 110 destroyed 100% of the microbial toxins during the 30 second exposure, as compared to the statistically nonsignificant drops in toxin reduction by water, or any of the other commercially available brands of oral rinses that were tested including chlorhexidine.

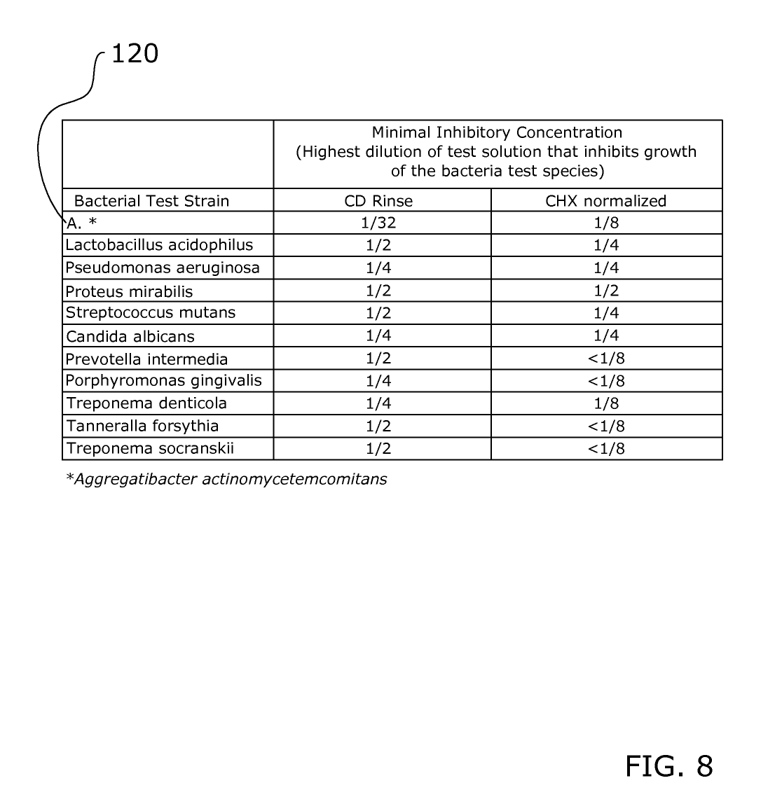

FIG. 8 is an exemplary table listing the comparative results of an 18 to 24 hour standard Minimum Inhibitory Concentration test of a chlorhexidine oral rinse solutions' effectiveness in inhibiting growth of bacteria typically found in the oral cavity, and during dental procedures. More specifically, under controlled test conditions of the University of Iowa College of Dentistry, May 15, 2012, a CD rinse solution was tested against and compared to the chlorhexidine (CHX) as shown.

The objective of the experiment was to determine the effectiveness of the CHX oral rinse to kill bacteria and a yeast that would typically be encountered in endodontic and periodontal procedures, and provide supporting data as to whether CD would produce statistically superior prognoses if used as a disinfecting and debriding solution in endodontic and periodontic procedures. Subsequent tests and analysis not shown proved that combined with acoustic agitation, and correspondingly the accelerated production of chlorine dioxide gas produced the most effective disinfecting method, resulting in the highest probability of successful patient outcomes from periodontal and endodontic procedures.

The first line reading of 1/32 120 indicates that the CD rinse was as effective at inhibiting growth of Aggregatibacter actinomycetemcomitans (“A”) at a dilution of 32 parts water to 1 part rinse as was a ⅛ dilution of chlorhexidine, illustrating the more effective nature of CD. On the other listed bacteria tested, the CD rinse did equally well or slightly less well than CHX. It should be noted that even when CHX outperformed CD, or performed as well as CD but at lower concentrations, it did so after continuously acting upon the bacteria for a continuous period of 18 to 24 hours; CD on the other hand while the CD tested had been active for only three minutes, yet remarkably continued to inhibit growth for the subsequent testing time period.

The three minutes during which CD was active against the texted microbial toxins is much more aligned with the typical duration of time during which disinfecting rinse solutions are applied. Thus, actual periodontal and endodontic procedures would be better served using a solution of CD as the results imply that fast-acting CD is a clinically preferred oral rinse over CHX.

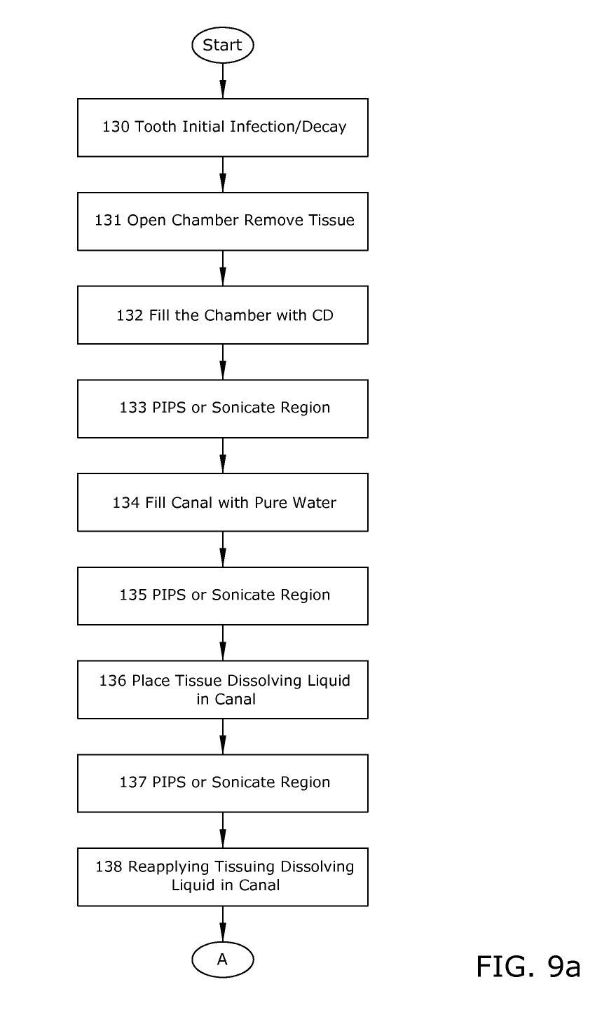

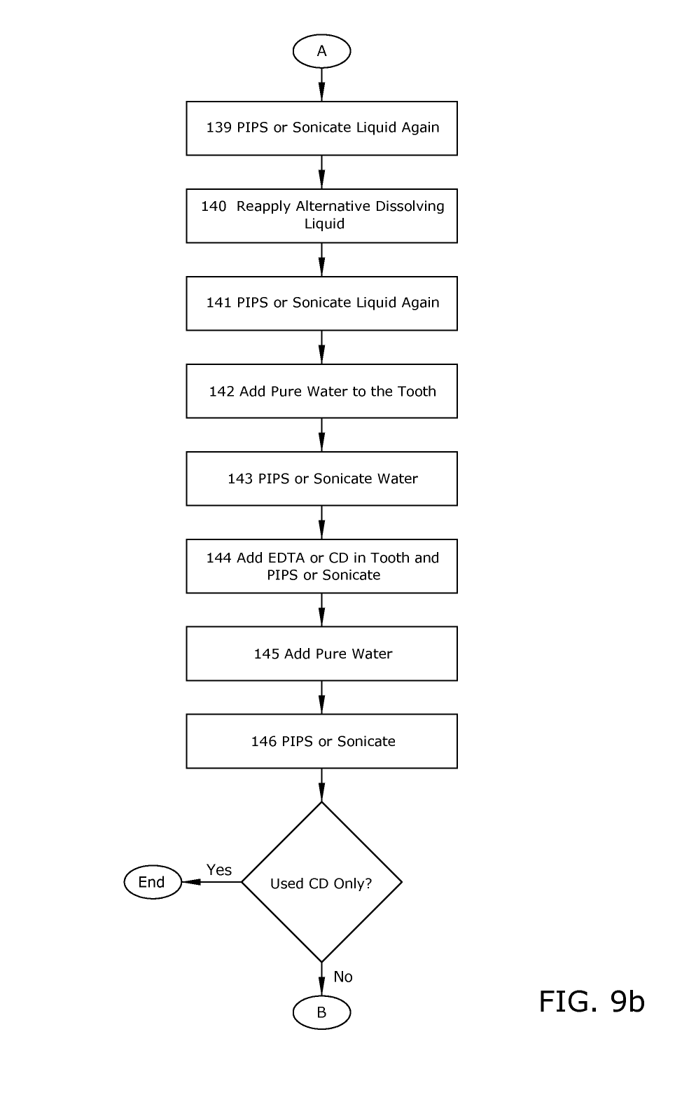

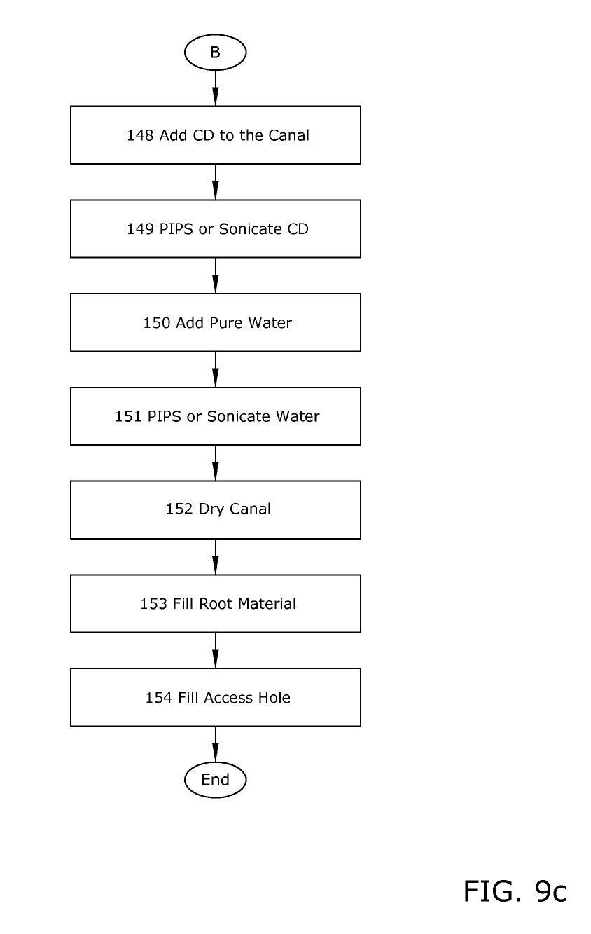

FIG. 9a-9c is an alternative exemplary flowchart illustration of a sequence of steps and the associated methods of performing root canal therapy to that found in FIG. 3. First a normal healthy tooth has an initial infection 130 of pulp chamber leading to tooth decay in the entire root canal system. A dentist opens 131 the tooth chamber for access to the root canal using instrumentation in the canal to remove initial bulk tissue and widen the canal. A dentist then fills 132 the root canal and chamber with CD. Next, one either PIPS or sonicates 133 the area for about 30 seconds and then fills 134 the canal and chamber with pure water. Then one PIPS or sonicate 135 for about 30 seconds once again and places 136 bleach or alternative tissue dissolving liquid into the canals and filling the chamber. PIPS or sonicate 137 activation of the bleach or alternative tissue dissolving liquid for about 30 seconds and then reapply 138 (a second) application of bleach or tissue dissolving liquid therein.

Once again PIPS or sonicate 139 the liquid again for about 30 seconds and then apply 140 a third application of bleach or alternative tissue dissolving liquid. Next, sonicate or PIPS 141 the bleach or tissue dissolving liquid a third time for about 30 seconds and then add pure water 142 to the tooth. Again, PIPS or sonicate 143 the water for about 30 seconds and add 144 EDTA or CD therein. Next, PIPS or sonicate 144 the EDTA or CD for about 30 seconds and add pure water 145; then one PIPS or sonicates 146 the area for about 30 seconds. The irrigation ends here 147 if you did not use EDTA and used CD only. However, if you did use EDTA on the last step before the pure water, now one adds 148 CD to the canals and chamber and then PIPS or sonicate 149 the CD for about 30 seconds. It should be understood that in this process CD should always be the last chemical irrigant before final flush with water. Then one adds 150 pure water to the region and PIPS or sonicate 151 the water for about 30 seconds and dries 152 the canals. A dental professional then fills 153 the root canals with an appropriate root canal filling material to seal them and fills 154 the access hole cut into the tooth with a restorative material.

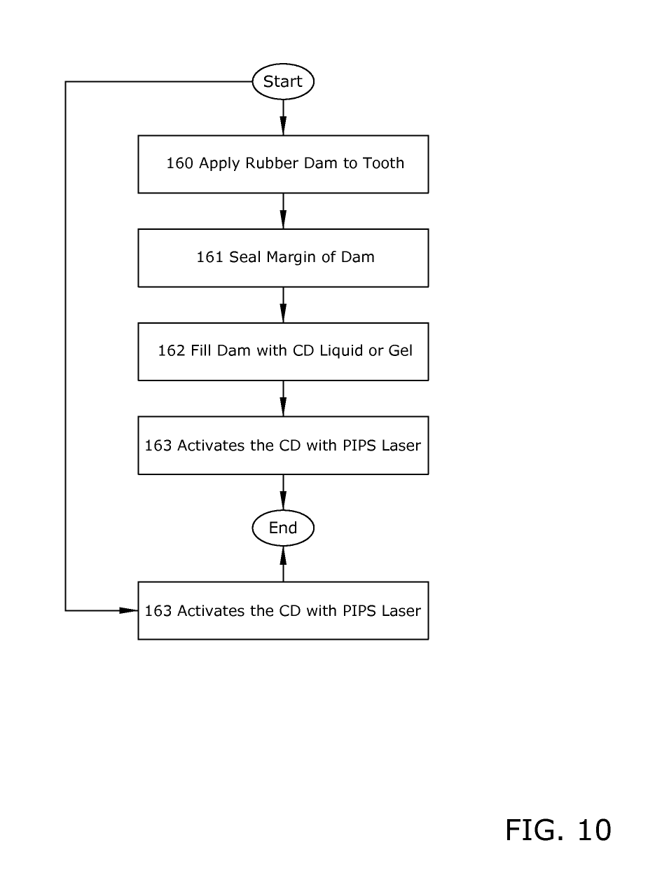

FIG. 10 is an alternative exemplary illustration of the therapeutic processes used to treat periodontal disease to that found in FIG. 5. First one applies 160 a rubber dam around the tooth with no interproximal rubber attachments. Then a dental professional seals 161 the margin of the rubber dam to the gum tissues with a liquid dam material whilst making sure that the rubber dam is not sealed against the tooth structure. Then one fills 162 the dam with CD liquid and/or CD gel to submerge all free gingival margins. Next a user activates 163 the CD with PIPS laser for three 30 second bursts or alternatively places 164 some form of PIPS laser tip, Magnetostrictive instrument or piezo instrument into the sulcus to activate the CD without the need for the rubber dam.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A method of cleaning a tooth comprising steps of:
preparing a tooth for irrigation by creating an orifice therein and removing tissues;
applying chlorine dioxide to an affected region of the tooth through the orifice in the tooth; and
activating the chlorine dioxide using an agitation tool.

2. The method claim 1, wherein the chlorine dioxide is comprised of a liquid state.

3. The method claim 1, wherein the chlorine dioxide is comprised of a gel state.

4. The method of cleaning a tooth of claim 3, wherein the applying chlorine dioxide step further comprises the step of filling the rubber dam with chlorine dioxide thereby submerging all free gingival margins.

5. The method claim 4, wherein the activating the chlorine dioxide using an agitation tool further comprises the step of activating the chlorine dioxide with a laser.

6. The method of claim 1, wherein the activating the chlorine dioxide using an agitation tool step further comprises the step of placing a tool tip into a sulcus of the tooth thereby activating the chlorine dioxide.

7. A method of sanitizing a diseased tooth having a dental surface comprising steps of:
opening the surface of the diseased tooth for access to an affected region therein;
removing tissue from the region so as to create a root canal; and
filling the region with chlorine dioxide.

8. The method claim 7, wherein the chlorine dioxide is comprised of a liquid state.

9. The method claim 7, wherein the chlorine dioxide is comprised of a gel state.

10. The method of claim 7, further comprising the steps of actuating the chlorine dioxide using an agitation tool.

11. The method of claim 10, further comprising the step of filling the region with pure water.

12. The method of claim 11, further comprising the step of activating an agitation tool proximate the affected region.

13. The method of claim 12, further comprising the step of placing a tissue dissolving liquid in the region.

14. The method of claim 13, further comprising the step of activating an agitation tool proximate the affected region.

15. The method of claim 14, further comprising the steps of repeating the placing a tissue dissolving liquid in the region and activating an agitation tool proximate the affected region two more times.

16. The method of claim 15, further comprising the steps of:
adding pure water to the region then activating an agitation tool proximate the affected region;
add a cleanser to the region then activating an agitation tool proximate the affected region;
adding pure water to the region; and
activating an agitation tool proximate the affected region.

17. The method of claim 16, further comprising the steps of adding a second cleanser to the region and activating an agitation tool proximate the affected region.

18. The method of claim 17, wherein the second cleanser is chlorine dioxide.

19. The method of claim 17, further comprising the steps of:
adding water to the region;
activating an agitation tool proximate the affected region;
drying the affected region;
filling the affected region of the tooth; and
filling an access hole.

20. A method for cleaning of a root canal in a diseased tooth, said method comprising the steps of:
removing tissue from a root canal chamber in a root of a diseased tooth;
adding chlorine dioxide to the root canal chamber; and
applying an activation tool to the chlorine dioxide in the root canal chamber.
